(19) United States
(12) Patent Application Publication
(10) Pub. No.: US 2020/0383557 A1
(43) Pub. Date: Dec. 10, 2020

(54) ENDOSCOPIC GRABBER WITH WIRELESS DEVICE AND COMPACT EXTENSIBLE CAMERA

(71) Applicant: Saunders Midwest LLC, Chicago, IL (US)

(72) Inventors: Walter J. SEDLACEK, West Chicago, IL (US); Douglas A. SPITLER, Naperville, IL (US); Michael R. BARTHEL, Prospect Heights, IL (US); Fule ZHANG, Shenzhen (CN)

(73) Assignee: Saunders Midwest LLC, Chicago, IL (US)

(21) Appl. No.: 16/769,901

(22) PCT Filed: Dec. 7, 2018
(86) PCT No.: PCT/US18/64519

§ 371 Date: Jun. 4, 2020

Related U.S. Application Data
(60) Provisional application No. 62/595,668, filed on Dec. 7, 2017.

(51) Int. Cl. A61B 1/018; A61B 17/29; A61B 1/005; A61B 1/00; A61B 1/05; A61B 8/12; G02B 23/24

(52) U.S. Cl. CPC A61B 1/018; A61B 17/29; A61B 1/005; A61B 1/00108; A61B 1/00147; A61B 2017/00734; A61B 1/05; A61B 1/0051; A61B 8/12; G02B 23/2476; A61B 1/00112

(57) ABSTRACT

An endoscopic grabber apparatus includes a proximal housing, a distal assembly, a flexible shaft extending between the proximal housing and the distal assembly, a flexible member within the flexible shaft having a distal end portion connected to the distal assembly and a proximal end portion connected to the proximal housing, and a camera within the distal assembly. A distal end portion of the flexible member is movably disposed within the distal assembly. A proximal end portion of the flexible member is connected to an actuator within the proximal housing. Actuation of the proximal end portion moves the distal end portion of the flexible member such that a protrusion extending from the distal end portion deforms to form part of a grabber. The camera is configured to capture an image including a tip portion of the protrusion. The endoscopic grabber device can include multiple protrusions configured to grab a target object

### CROSS-REFERENCE TO RELATED APPLICATIONS

This application claims benefit of priority to U.S. Provisional Application Ser. No. 62/595,668, entitled “ENDOSCOPIC GRABBER WITH WIRELESS CAMERA AND COMPACT EXTENSIBLE CAMERA,” filed Dec. 7, 2017, which is incorporated herein by reference in its entirety.

### BACKGROUND

The embodiments described herein relate to grabbing tools and extensible viewing devices. More specifically, embodiments described herein relate to grabbing tools having viewing capabilities including hand-operated grabbing tools combined with one or more endoscopic cameras, as well as to compact extensible viewing devices.

Known grabbing tools for allowing operators to reach, grab, and/or interact with an object via a hand-operated assistive devices are directed to performing motor functions alone, such as grabbing an object, without providing additional functionality. These devices rely on the user being able to view the target object sufficiently well with the naked eye to reach and manipulate the object, as well as the nearby environment sufficient well to navigate to the object. These tools include tools to grab items located beyond the user's reach, such as items located in tight or hard-to-reach locations. These tools also include extensible tools, such as wrenches, screw drivers and magnetic tips located at the end of an extension.

Many known tools for allowing operators to view a target area, such as endoscopes or borescopes, are similarly directed to performing viewing functions alone without providing additional functionality. These devices permit a user to guide a lens or camera to a hard-to-reach area and either take pictures of the target area or send video signals to viewer device seen by the user. Such known tools include camera mounts and endoscopic viewing devices. However, such known devices often provide only viewability of the target object or target area. As such, the user is required to perform subsequent actions to interact with an object viewed initially using the viewing device, such as to grab or manipulate a target object thereafter using a second tool without viewability assistance.

Further, many known viewing or inspection tools are overly complicated or expensive and are not easily combined with an assistive tool, such as with a grabber tool. Conventional viewing tools include expensive, specially designed electronic devices that provide particular types of viewing functions. For example, some known conventional medical endoscopes are designed to provide customized viewing for medical diagnoses or treatments. However, they are not well suited, and would be overly expensive to use, for both viewing a target object and manipulating the object with an extensible assist tool, such as to find a dropped bolt while working on a device. As another example, conventional industrial viewers are known that are designed for inspecting the integrity of a structure or for evaluating a necessary repair in construction industries. Likewise, these devices are not well suited, and would be overly expensive to use, for viewing a common or household target object to grab with a grabber tool.

Thus, a need exists for improved grabber tools, devices, and methods that provide viewing, lighting, or sensing features in combination with motility features of a grabber tool. Further, a need exists for simple and inexpensive devices for providing viewability in hard-to-reach locations for a user as needed without specialized viewing devices.

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter.

In some embodiments, an apparatus, such as an endoscopic grabber apparatus, includes a proximal housing including an actuator, a distal assembly, a flexible shaft, and a flexible member movably disposed within the flexible shaft. The distal assembly includes an outer housing, an inner housing, a set of protrusions, and an electronic device. The inner housing is movably disposed within the outer housing, and the inner housing defines a bore. The set of protrusions is coupled to an outer surface of the inner housing such that at least a portion of each protrusion is within a volume between the outer surface of the inner housing and an inner surface of the outer housing. The electronic device is coupled within the bore of the inner housing. A proximal end portion of the flexible shaft is coupled to the proximal housing and a distal end portion of the flexible shaft is coupled to the outer housing. A distal end portion of the flexible member is coupled to the inner housing, and a proximal end portion is coupled to the actuator. The actuator is configured to move the flexible member to cause the inner housing to move within the outer housing between a first position and a second position. The distal end portion of each protrusion from the plurality of protrusions is in a first configuration within the outer housing when the inner housing is in the first position, and is in a second configuration outside of the outer housing when the inner housing is in the second position. A distal tip of each protrusion from the plurality of protrusions extends outside of the outer housing when the inner housing is in the second position.

In some embodiments, the electronic device is a camera, a light emitting device, or an ultrasonic device. In other embodiments, the electronic device can be any sensing device, such as an infrared sensor, an optical sensor, a temperature sensor, pressure (e.g., sound pressure level) sensor, a biological sensor, a gas sensor, a radiation sensor, or the like. In some embodiments, the electronic device can include a wireless network interface configured to transmit a short range wireless signal associated with an image or a signal received and/or produced by the electronic device.

In some embodiments, an endoscopic grabber apparatus includes a proximal housing, a distal housing, and a flexible shaft extending between the housings and connected to each housing. The endoscopic grabber apparatus further includes a flexible member within the flexible shaft having a distal end portion connected to the distal housing, and a proximal end portion connected to the proximal housing. The flexible member further includes a proximal end portion coupled to an actuator disposed within the proximal housing. Actuation of the proximal end portion of the flexible member moves a distal end portion of the flexible member within the distal housing to deform a protrusion extending from the distal end portion. The apparatus includes a camera within the distal housing that can receive an image that includes an end portion of the protrusion. In some embodiments, the camera includes an optical sensor within the distal housing coupled with an electronic module within the proximal housing. In some embodiments, the electronic module is coupled with a power source and a wireless interface configured to transmit the image to an electronic device.

In some embodiments, the actuation of the proximal end portion produces linear movement of the distal end portion. The linear movement of the distal end portion radially deforms a tip portion of the protrusion to rotate away from a longitudinal direction of the protrusion prior to the actuation. In some embodiments, the endoscopic grabber includes a plurality of protrusions, and actuation of the distal end portion radially deforms tip portions of the plurality of protrusions. The tip portions of the plurality of protrusions can grab a target object in a central region disposed between the protrusions. In some embodiments, the camera image can include the tip portions of the plurality of protrusions. In some embodiments, a line segment can be determined that extends between each of the tip portions to a central point located between the tip portions, and the camera image can show the line segments in the camera image as virtual line segments. In some embodiments, the virtual line segments can be shown as virtual cross-hairs to help guide a user during grabbing or manipulation operations for the target object.

In some embodiments, a compact extensible camera device includes an extensible handle portion and a camera portion. The extensible handle portion can be arranged to move between a retracted, compact position and a plurality of extended positions. The camera portion can be attached and removed from the extensible handle portion and can include internal storage for storing images captured by the camera. In some embodiments, the extensible handle portion can be formed as a telescoping handle having a plurality of nested segments concentrically disposed within each other in a telescoping arrangement.

Other devices, systems, components, features, implementations, methods, and/or products according to embodiments will be or become apparent to one with skill in the art upon review of the following drawings and detailed description. It is intended that all such additional devices, systems, components, features, implementations, methods, and/or products be included within this description, be within the scope of this disclosure.

### BRIEF DESCRIPTION OF THE DRAWINGS

FIG. 1 is a perspective view of an endoscopic grabber device according to an embodiment.

FIG. 2 is a front perspective view of the embodiment of FIG. 1 showing the endoscopic grabber in an extended position.

FIG. 3 is a side view of the embodiment of FIG. 1 showing the endoscopic grabber in an extended position.

FIG. 4 is a side view of the extended position of the endoscopic grabber of FIG. 3 shown with the distal housing removed.

FIG. 5 is a side view of the embodiment of FIG. 1 showing the endoscopic grabber in a retracted position.

FIG. 6 is a side view of the retracted position of the endoscopic grabber of FIG. 5 shown with the distal housing removed.

FIG. 7 is a side view of the endoscopic grabber of FIG. 1 showing the endoscopic grabber in a retracted position.

FIG. 8 is lengthwise cross-sectional view of the endoscopic grabber of FIG. 7 showing the endoscopic grabber in a retracted position.

FIG. 9 is a lengthwise cross-sectional view of the endoscopic grabber of FIGS. 1-7 showing the endoscopic grabber in an extended position.

FIG. 10A is an enlarged view of the distal end portion of the endoscopic grabber shown in FIG. 9.

FIG. 10B is a zoomed view of region K indicated in FIG. 10A of a portion of the distal end portion of the endoscopic grabber shown in FIG. 9.

FIG. 10C is cross-sectional view of a portion of the distal end portion of the endoscopic grabber shown in FIG. 9 as viewed from line X-X shown in FIG. 10B.

FIG. 10D is an enlarged view of the proximal end portion of the endoscopic grabber shown in FIG. 9.

FIG. 11 is a front view of a viewer device that can be used with the endoscopic grabber device shown in FIGS. 1-10D according to an embodiment, which shows an example camera view from the endoscopic grabber.

FIG. 12A is a side view of an endoscopic grabber according to an embodiment in a retracted position, which is shown with the flexible shaft following an example curvilinear path.

FIG. 12B is a side view of a portion of the endoscopic grabber of FIG. 12A in a retracted position, in which the flexible shaft follows an example linear path.

FIG. 12C is a lengthwise cross-sectional view of the proximal end portion of the endoscopic grabber of FIG. 12A.

FIG. 13 a perspective view of a compact extensible camera according to an embodiment.

FIG. 14 is a side perspective view of the compact extensible camera of FIG. 13.

FIG. 15 is a rear perspective view of the compact extensible camera of FIG. 13.

FIG. 16 is a front perspective view of the compact extensible camera of FIG. 13.

FIG. 17 is top perspective view of the compact extensible camera of FIG. 13.

FIG. 18 is a perspective view of the camera portion of the compact extensible camera of FIG. 13.

FIG. 19A is a left side view of the camera portion shown in FIG. 18.

FIG. 19B is a front view of the camera portion shown in FIG. 18.

FIG. 19C is a top view of the camera portion shown in FIG. 18.

FIG. 19D is bottom view of the camera portion shown in FIG. 18.

FIG. 19E is rear view of the camera portion shown in FIG. 18.

FIG. 19F is a bottom view of the camera portion shown in FIG. 18.

### DETAILED DESCRIPTION

The embodiments described herein can advantageously be used in a variety of endoscopic grabber devices and compact extensible camera devices, tools and components, and associated methods and operations. In particular, the devices described herein can be integrated endoscopic grabber and extensible viewer devices, accessories and components for viewing target objects in difficult to reach locations, as well as for grabbing or manipulating target objects while concurrently viewing the objects and/or the corresponding environments.

Various example features, aspects, configurations, components, assemblies, and arrangements are generally described herein pertaining to an endoscopic grabber device, such as example endoscopic device 100, which can be used to grab and/or manipulate a target object while also viewing, lighting, and/or sensing the object and corresponding environment. Embodiments of endoscopic grabber devices described herein are each configured to operate as an integrated endoscopic grabber device that an operator can use to reach, grab, and optionally manipulate a target object while concurrently viewing the object and nearby environment. The user can simply maneuver a distal grabber portion of the endoscopic grabber device into a position close to the target object with the aid of concurrent views from a camera disposed on the distal grabber portion. Further, the user can simply actuate the proximal handle portion of the endoscopic grabber device when positioned with respect to the target object to operate the distal grabber portion to grab and/or manipulate the target object. The grabbing and/or manipulation operations regarding the target object can be greatly enhanced by providing the user with concurrent views from the camera.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

The term “flexible” in association with a part, such as a mechanical structure, component, or component assembly, should be broadly construed. In essence, the term means the part can be repeatedly bent and restored to an original shape without permanently deforming the part. Certain flexible components can also be resilient. For example, a component (e.g., a flexure) is said to be resilient if possesses the ability to absorb energy when it is deformed elastically, and then release the stored energy upon unloading (i.e., returning to its original state). Many “rigid” objects have a slight inherent resilient “bendiness” due to material properties, although such objects are not considered “flexible” as the term is used herein.

As used in this specification and the appended claims, the word “distal” refers to direction towards a target object, and the word “proximal” refers to a direction away from the target object. Thus, for example, the end of an endoscopic grabber device that is closest to the target object or target surface would be the distal end of the endoscopic grabber device, and the end opposite the distal end (i.e., the handle end manipulated by the user) would be the proximal end of the endoscopic grabber device.

Further, specific words chosen to describe one or more embodiments and optional elements or features are not intended to limit the invention. For example, spatially relative terms—such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes includes various spatial device positions and orientations.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

As used herein, the term “camera” in the context of an electronic device refers to an electronic optical device for a capturing an image, which can include one or more sensor components for receiving the image and/or one or more components for interpreting, transforming, managing, storing or otherwise processing the image to be in a viewable format. As such, a camera can include one or more components separated from each other, such as an electronic image sensor at a first location that captures an image, and an electronic control module or other processing component for processing the captured electronic image into a viewable format, which can be at a second location that is collocated with or spaced apart from the first location.

Unless indicated otherwise, the terms apparatus, device, tool, etcher and variants thereof, can be interchangeably used.

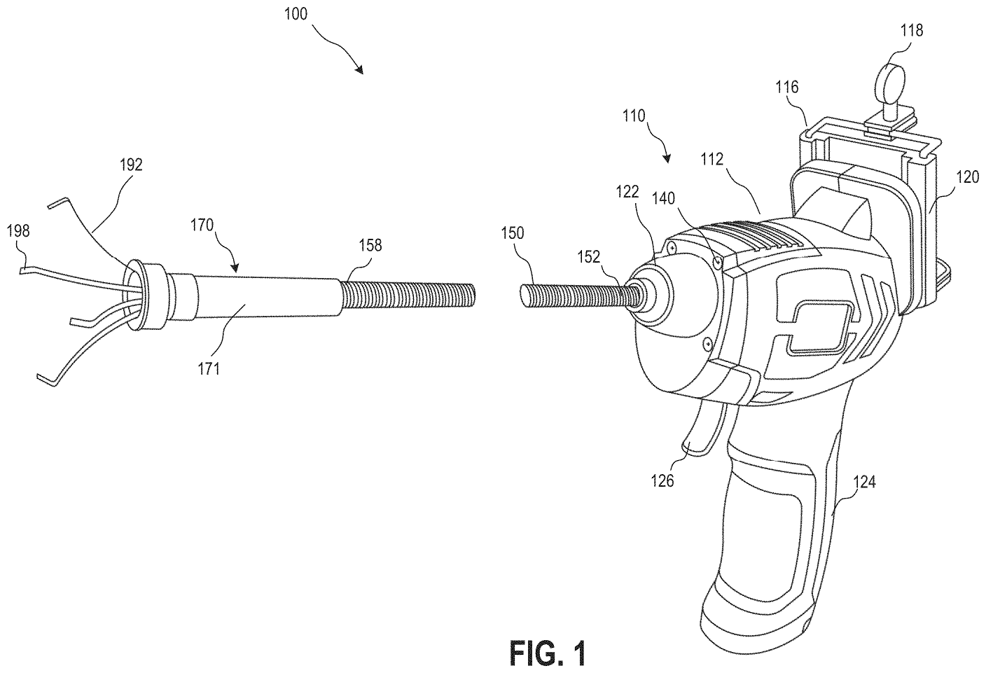

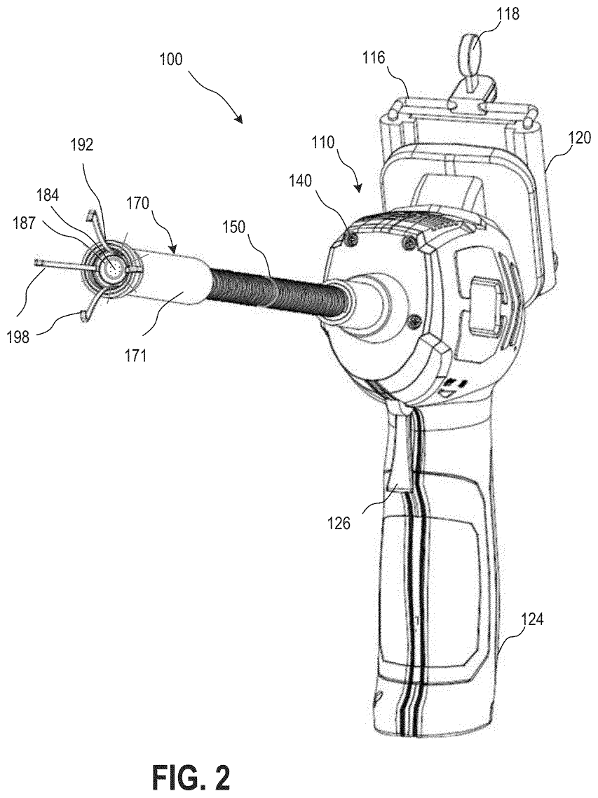

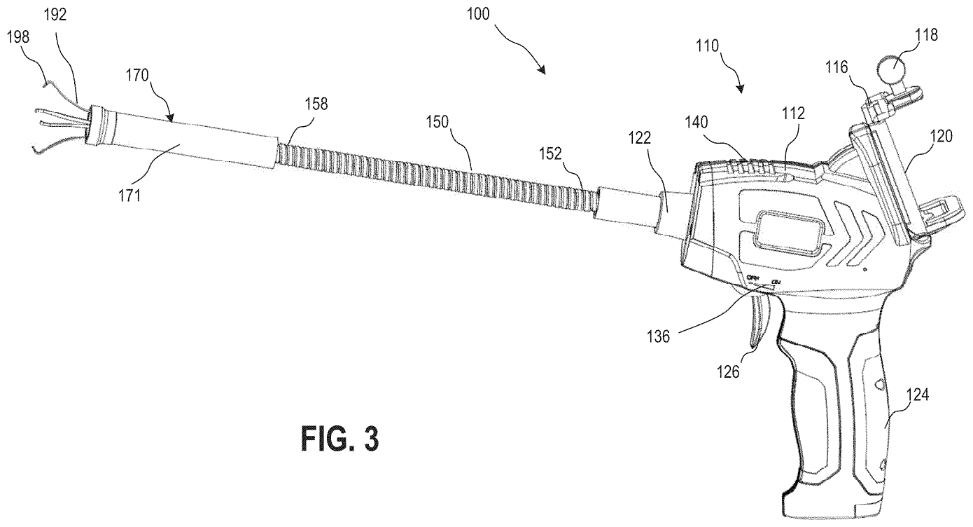

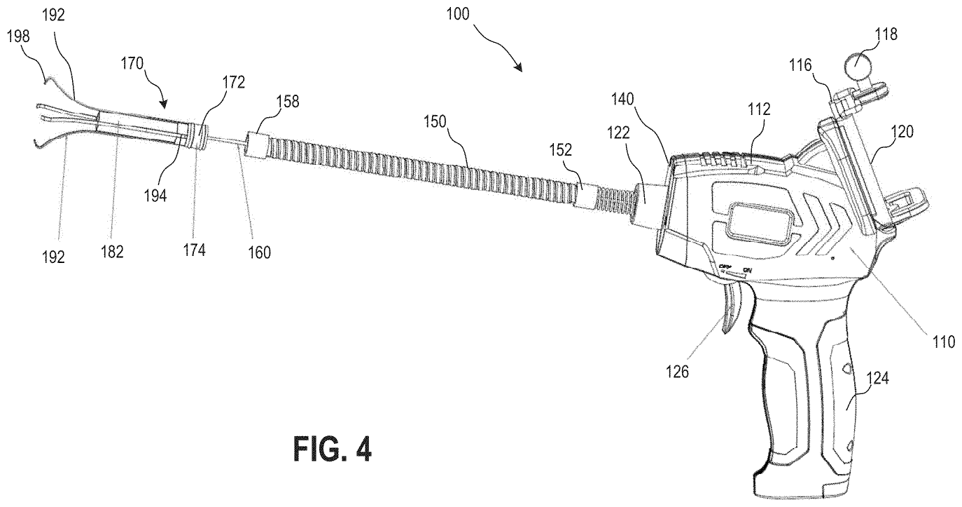

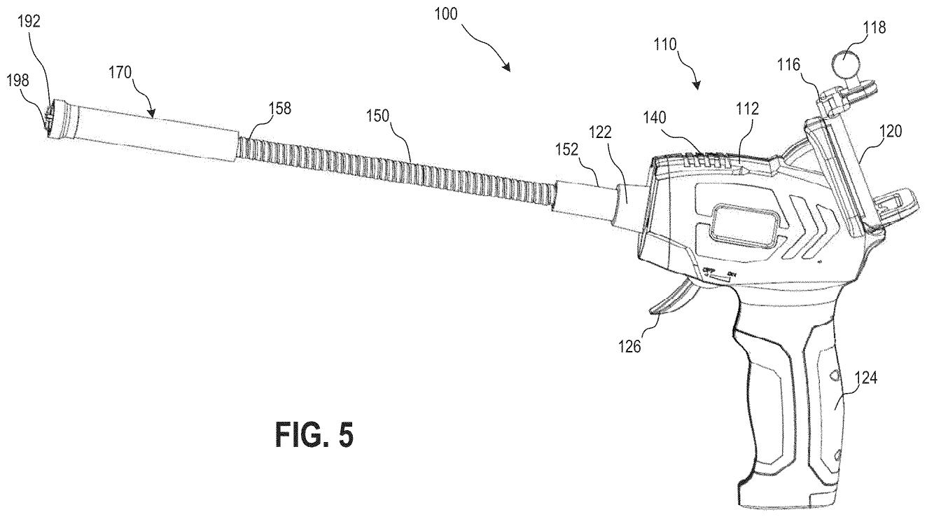

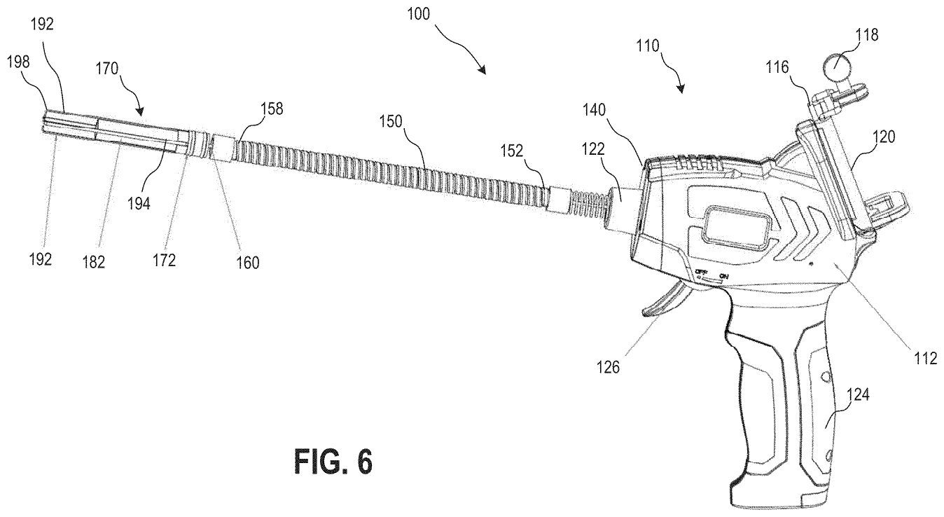

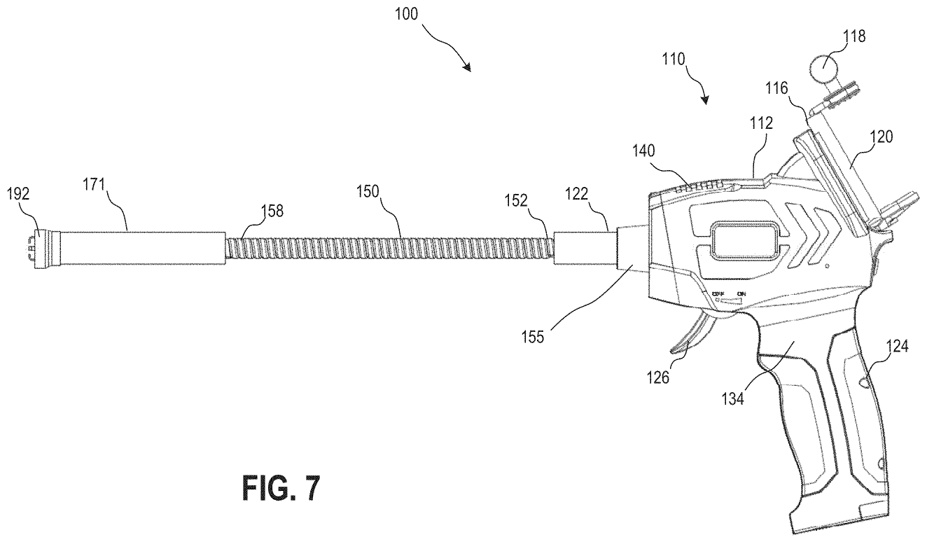

FIGS. 1-10D show an example endoscopic grabber device 100 according to an embodiment. The endoscopic grabber device 100 includes a control assembly 110 having a proximal housing 112 and the control components therein, a distal assembly 170, a flexible shaft 150 extending between the control assembly 110 and the distal assembly 170, and a flexible member 160 located within the flexible shaft 150. The flexible shaft 150 has a distal end portion 158 connected to the distal assembly 170, and a proximal end portion 152 connected to the proximal housing 112. The control assembly 110 includes an actuator 126 coupled to the proximal housing 112 that is configured to actuate movement of the flexible member 160 as described herein. The flexible member 160 is movably disposed within the flexible shaft 150. A distal end portion 168 of the flexible member 160 is coupled to the distal assembly 170, and a proximal end portion 162 of the flexible member is coupled (via a mounting end 156) to the actuator 126 of the proximal housing 112. The actuator 126 is configured to move the flexible member 160 to actuate movement of the distal assembly 170.

The flexible member 160 can be formed as a flexible wire. The flexible shaft 150 can be formed from a flexible metal shaft, a flexible elastomeric shaft, or the like, and defines an internal channel for the flexible member 160 to translate therein while permitting the shaft to bend and flex as needed to reach a target object during use. The flexible shaft 150 further includes the mounting member 156 or push wire mounting member 156 and a spring 154 disposed at its proximal end portion 152. The spring is retained within the proximal end portion 152 and biases the flexible member 160 forward in the direction of the proximal housing 112. As such, actuation of the actuator 126 acts to move the mounting member 156 against the bias of the spring 154, and the spring urges the mounting member and flexible member 160 rearward toward the distal housing 170 when released. Thus, actuation of actuator/trigger 126 advances the flexible member forward into an extended arrangement, and releasing the actuator/trigger 126 biases the flexible member to rearward into an extracted arrangement.

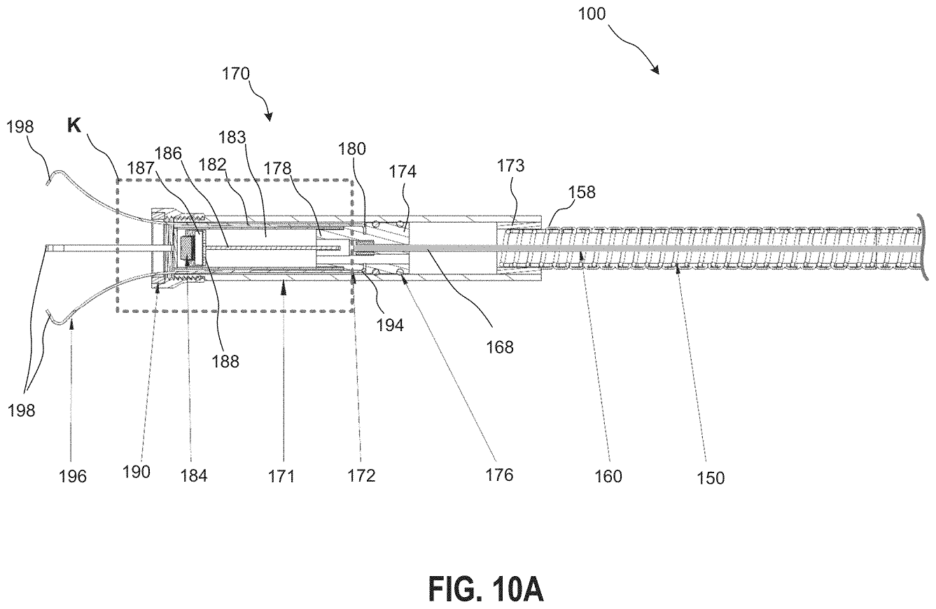

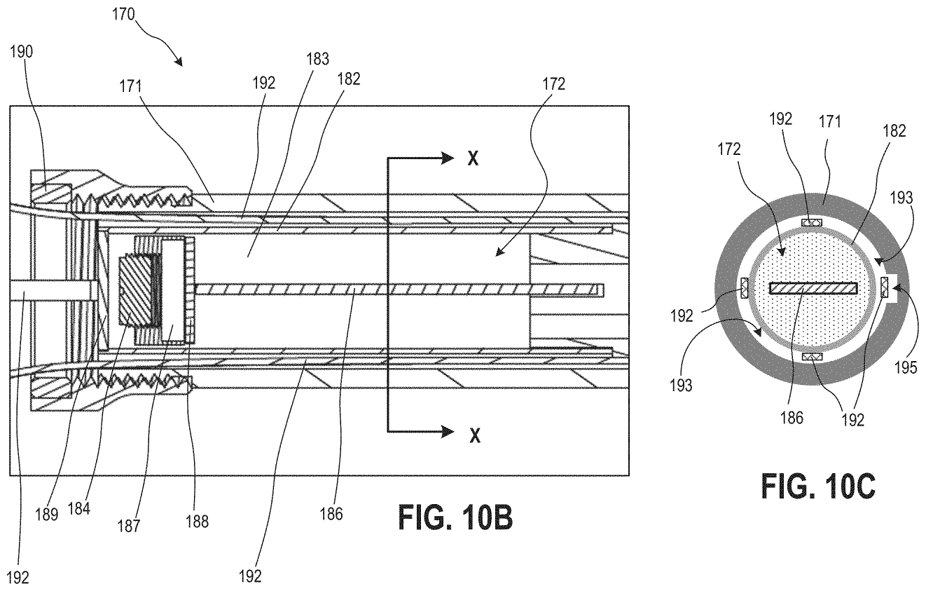

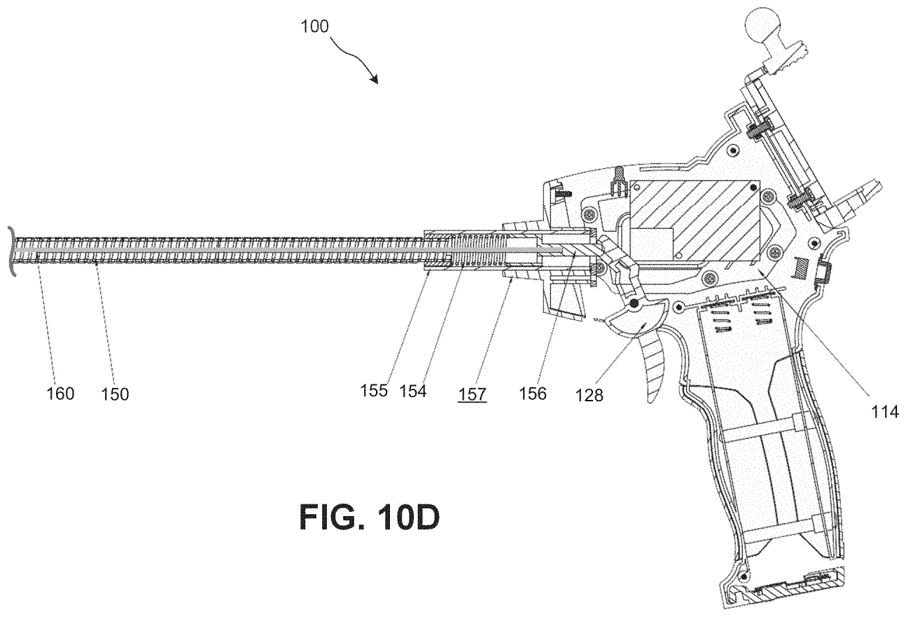

Referring to FIGS. 10A-10C, the distal assembly 170 includes an outer housing 171, an inner housing 182 movably disposed within the outer housing, a plurality of protrusions 192 coupled to an outer surface of the inner housing 182, and an electronic device 184. A coupling portion 173 at a proximal end portion of the outer housing 171 connects the distal end portion 158 of the flexible shaft to the outer housing. In this manner, the outer housing 171 remains fixedly coupled to the flexible shaft 150 during operation of the device 100. Similarly stated, when the flexible member 160 is actuated, the outer housing 171 does not move relative to the flexible shaft 150.

The outer housing 171 defines an interior volume within which the inner housing 182 is located. As shown in FIG. 10C, the outer housing 171 can be a tubular housing such that the interior (or inner surface) of the outer housing is defined by an internal diameter of the outer housing 171. An exterior portion (or outer surface) of the inner housing 182 (and/or the movable member 172) is smaller than the interior of the outer housing such that the inner housing 182 fits within the outer housing 171. As such, the inner housing 182 is configured to move or translate within the outer housing 171 and, thereby, operate as a portion of a movable carrier for the distal assembly 170, as described below. More specifically, referring to FIG. 10C, an annular gap 193 within which the protrusions 192 are maintained is defined between the exterior of the inner housing 182 and the interior of the outer housing 171. In some embodiments the inner surface of the outer housing 171 can define one or more guide channels 195. Such guide channels can have an elongate orientation (i.e., can extend along a longitudinal axis of the distal assembly 170) to guide each of the protrusions 192 during actuation and movement between the first and second configurations. For example, FIG. 10C shows a single guide channel 195 oriented in an elongate orientation with one of the protrusions 192 as an example to illustrate the optional use of guide channels. In other embodiments, the outer housing 171 does not define any guide channels, which arrangement allows the inner housing 182 and/or the protrusions 192 to rotate relative to the outer housing 171 about the longitudinal axis of the distal assembly 170. In other embodiments, the outer housing 171 can include multiple guide channels (e.g., one for each of the protrusions 192).

The distal end portion of the outer housing 171 includes a magnetic attachment member (also referred to as the magnet) 190 that can assist with attracting or coupling to a target object. Although the magnet 190 is shown as being threadedly coupled to the distal end portion of the outer housing 171, in other embodiments, a magnet can be coupled to the outer housing 171 by any suitable means, such as by a press fit, an adhesive, or the like. In yet other embodiments, the outer housing 171 need not include the magnetic attachment member 190.

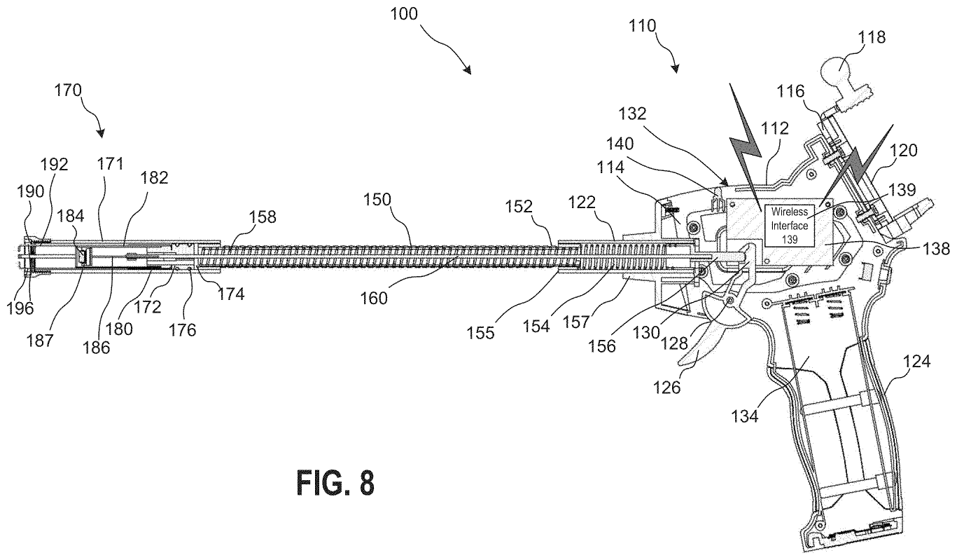

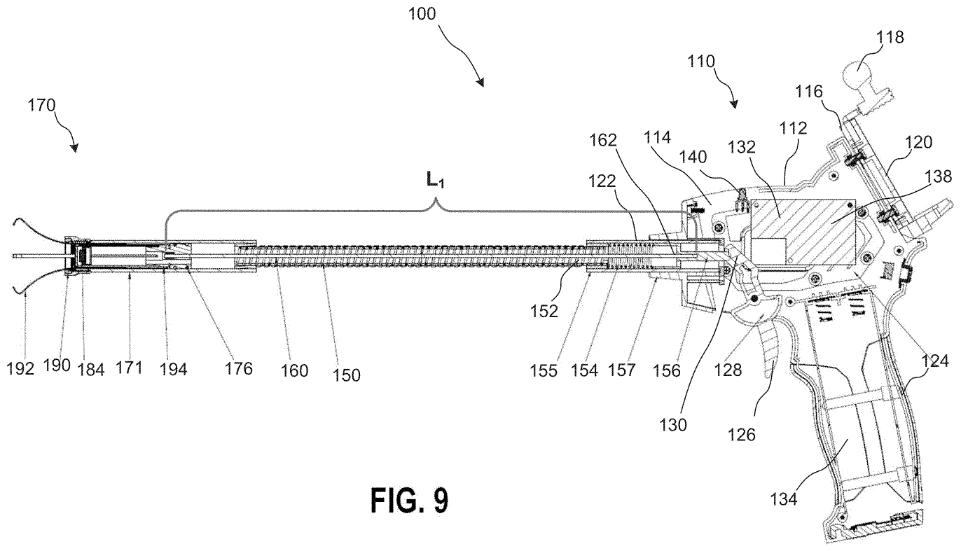

As further shown in FIG. 10C, the interior of the inner housing 182 defines a bore 183, within which the electronic device 184 is coupled. The proximal end portion of the inner housing 182 includes or is coupled to a movable member 172 (which functions as a junction member between the flexible member 160, the electronic device 184, and the protrusions 192). As shown in FIG. 10A, a distal end portion 178 of the movable member 172 is aligned with and coupled to a proximal end portion of the inner housing 182 within the interior of the outer housing 171. As such, the movable member 172 and the inner housing 182 operate together to form a translatable movable carrier (e.g., to carry the electronic device 184 and/or the protrusions 192) within the outer housing 181. Although the inner housing 182 and the movable member 172 (or junction member) are shown as being separate components that are joined together, in other embodiments, the inner housing 182 and the movable member 172 can be monolithically constructed. A proximal end portion 174 of the movable member 172 is coupled to a distal end of the flexible member 160, which extends from the distal end 158 of the flexible shaft and into the interior of the outer housing 171, to couple to the proximal end portion of the movable member. As such, the flexible member 160 extends between the movable member 172 and the actuator 126 through the flexible shaft 150, such that movement of the actuator 126 causes the inner housing 182 and the movable member 172 to move together within the outer housing 181 between a first position (FIG. 8) and a second position (FIGS. 9 and 10A).

As noted above, the movable member 172 joins the inner housing 182 to the flexible member 160, the electronic device 184, and the protrusions 192. Specifically, the proximal end portion 174 of the movable member 172 includes a protrusion connection 180 through which the protrusions 192 are coupled to the movable member 172. Although the protrusion connection 180 shows a portion of each protrusion 192 being embedded within the movable member 172, in other embodiments, the protrusions 192 can be coupled to the movable member 172 and/or the inner housing 182 by any suitable mechanism (e.g., by a weld joint, an adhesive joint, or the like). In some embodiments, the protrusions 192 can be monolithically constructed with the movable member 172. The proximal end portion 174 of the movable member is also attached to the distal end 168 of the flexible member 160 and includes sealing rings 176. The sealing rings 176 are disposed around the movable member 172 within the bore 182 of the distal housing retain the movable member 172 in the sliding arrangement within the annular volume 193 defined within the outer housing 171. The sealing rings 176 can be formed from polymeric materials that provide a low-friction connection within the distal housing to enable sliding movement therein and that also prevent dust, dirt or other foreign materials from entering the interior of the outer housing 171.

As shown in FIG. 10B, the electronic device 184 is attached to the distal end portion 178 of the movable member 172 and is oriented to produce light, capture images, and/or sense conditions when the device is actuated. The electronic device 184 includes a mounting member 188 and electrical connections 186 that are coupled to the movable member 172. The mounting member 188 can include any suitable mechanism for securing the electronic device 184 within the bore of the inner housing 182. For example, in some embodiments, the mounting member 188 can include shock-absorbing properties, an interference fit portion, or any other suitable features to retain the electronic device 184 at the desired position within the inner housing 182. The electrical connections 186 can include one or more wires that electrically connect the electronic device 184 to the electronic controller 138. The wires can be collocated with the flexible member 160 and can extend through the flexible shaft 150 along with the flexible member. The wires (along with the electrical connections 186) can allow power to be conveyed from the battery 134 to the electronic device 184. The wires (along with the electrical connections 186) can also allow control signals and/or data signals to be transferred between the electronic device 184 and the electronic controller 138.

The electronic device 184 can be any suitable device that can produce light and/or sense conditions adjacent the distal end of the device 100. For example, in some embodiments, the electronic device can be a camera, a light emitting device, or an ultrasonic device. In other embodiments, the electronic device can be any sensing device, such as an infrared sensor, a temperature sensor, a radiation sensor, a gas sensor, or an optical sensor. In some embodiments, the electronic device 184 can include (or be coupled to) a wireless network interface configured to transmit a short range wireless signal associated with an image or a signal received and/or produced by the electronic device 184.

As shown in FIG. 10A, each of the protrusions 192 includes a proximal end 194 and a distal end 196. Each of the distal ends includes a distal tip 198. Referring to FIGS. 8 and 10A-10C, as described above, the proximal end 194 of each of the plurality of protrusions 192 is coupled to an outer portion of the movable member 172 at the coupling portion 180 of the movable member. Each of the protrusions extends from the respective proximal end 194 to the respective distal end 196 of the protrusion. As can be seen in FIG. 8, the distal end 196 of each protrusion 192 from the plurality of protrusions is configured to be at first position within the outer housing 171 when the inner housing is in a first, non-deployed position. When the protrusions 192 are in the first position, the distal tip 198 of each protrusion extends from the distal-most surface of the outer housing 171 by a first distance. In other embodiments, however, the distal tip 198 of each protrusion can be fully retracted within the outer housing 171 when the protrusions are in the first position. Moreover, when the protrusions 192 are in the first position, they are also in a first (deformed) configuration. Specifically, the each of the protrusions is within the annular volume 193 and is therefore deformed by the inner housing 182 and the outer housing 171 to be in a substantially linear configuration. Similarly stated, when in the retracted position, each of the protrusions 192 extends in a longitudinal direction that is generally parallel with the longitudinal axis of the distal assembly 170.

As shown in FIG. 10A, when the device 100 is actuated, the distal tip 198 of each protrusion 192 from the plurality of protrusions is at a second position extending outside of the outer housing 171. More specifically, because the protrusions 192 are coupled to the inner housing 182 (via the movable member 172), when the movable member 172 and inner housing 182 are moved within the outer housing 171, the protrusions 192 relative to the outer housing 171 to deploy the protrusion 192. When the protrusions 192 are in the second (deployed) position, the distal tip 198 of each protrusion extends from the distal-most surface of the outer housing 171 by a second distance, greater than the first distance. The protrusions 192 are each made from a flexible material (e.g., spring steel) that is arranged to rotate or flex outward away from each other and away from their longitudinal direction as they are translated forward out of the bore during actuation of the device 100. The distal ends 196 are arranged to form a set of inward directed hook-like shapes at each tip 198. As such, when the actuator 126 is actuated, the tip 198 of each protrusion 192 is directed inward toward a central region disposed the protrusions and a central point between the tips. In this manner, when the device 100 is returned to its undeployed state, the tips 198 can grasp an object within the central region.

In addition to moving the protrusions 192 between their first position and their second position, actuation of the device 100 also move the electronic device 184 between its first position within the outer housing 171 to its second position within the outer housing 171. Specifically, because the electronic device 184 is fixedly coupled within the inner housing 182, movement of the inner housing 182 and the movable member 172, which causes movement of the protrusions 192, also causes the electronic device 184 to be moved outward from its first (inward) position to its second (outward) position.

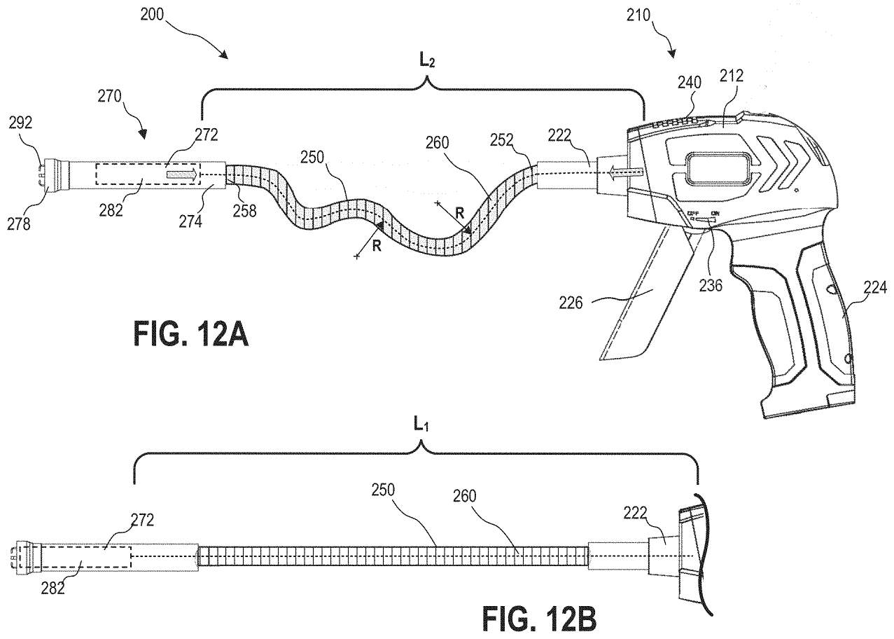

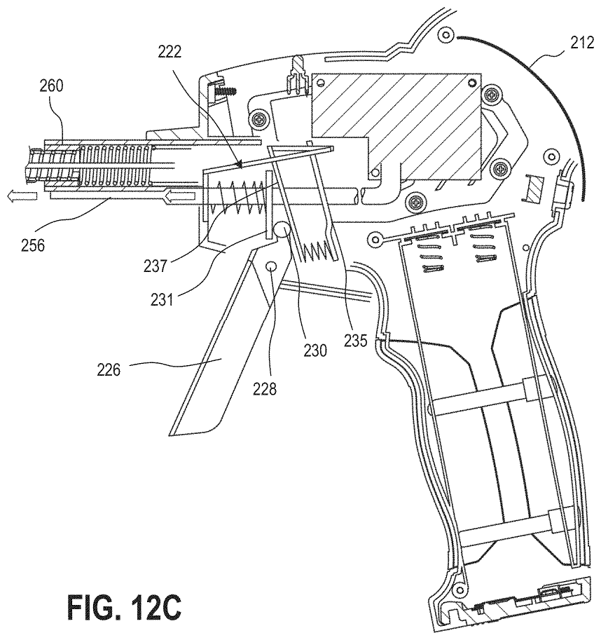

The relative position of the electronic device 184 and the tips 198 when the protrusions are deployed can cooperatively function to provide advantageous data collection. For example, as described herein, in embodiments in which the electronic device 184 is a camera, the camera can receive an image that includes an end portion 196 of the protrusions. In addition, the electronic control 132 can be configured to identify the tips 198 in the image. The electronic control 132 can further be configured to identify the central point between the tips and a line segment between each tip and the central point, and to show the line segments and/or central point as virtual features in the display device as discussed further below along with FIG. 12.

As best seen in FIGS. 8, 9 andl OD, the control assembly 110 includes the proximal housing 112, a rear handle or grip portion 124, a shaft connection 155, a manipulator portion 122 that receives the proximal portion of the flexible member 160, an actuator or trigger 126, and an electronics module 132. The proximal housing 112 defines an internal volume 114 therein, in which the electronics module 132 is secured. The proximal housing further includes a viewer mounting portion 116 that is arranged to removably retain a viewing device or phone device, such as viewing device 200 shown in FIG. 12. The mounting portion 116 includes a flat face 120 for receiving the viewing device 200, and a mounting knob 118 for removably retaining the viewing device during use.

The rear handle or grip portion 124 is configured as a handle that can be easily held by a user and allow the user to manipulate the endoscopic grabber device 100 during use. The actuator or trigger 126 is disposed on an upper, front region of the grip portion 124 and located for easy access by a user's index finger. The actuator or trigger 126 includes a pivot portion 128 that is rotatably mounted within the internal volume 114 of the proximal housing. A lever end 130 of the actuator or trigger 126 is located on an internal end of the pivot portion 128 adjacent to a mounting end 156 at the proximal end portion 162 of the flexible member 160. The lever end 130 is arranged in a cantilever arrangement with the exposed trigger end of the actuator 126 on the pivot portion 128. Actuation of the trigger end of the actuator 126 rotates the pivot portion 128 to move the lever end 130 to rotate away from the proximal housing toward the distal housing 170 and push the flexible member 160 to translate forward within the flexible shaft 150 in the direction of the distal housing 170. A face of the lever end 130 can be curved to maintain good contact with the mounting end 156 of the flexible member 160 during actuation.

The shaft connection 155 is disposed on a forward portion of the proximal housing 112 to securely connect the proximal end portion 152 of the flexible shaft 150 to the proximal housing. The manipulator portion 122 is disposed within the shaft connection 155 to receive the proximal end portion 152 of the flexible shaft 150 and to retain components of the proximal end portion of the flexible shaft, which are discussed in more detail below along with the flexible shaft.

The electronics module 132 includes a power source (or battery, not shown), a control switch 136, a controller 138 (which can include a processor), and one or more lights 140. As shown in FIGS. 8, 9 andl OD, the power source can include a battery within a battery storage region 134 defined within the proximal housing. In other configurations, the power source can include components for coupling to an alternating current power supply (not shown) in addition to components for a battery power source or as an alternative to a battery power source. Such configurations can include a power cord and transformer, as well as a charger for charging a battery. The control switch 136 can include a simple on/off switch, as well as optional settings for activating the lights 140 and/or the electronic device 184. The controller 134 can include a processor, a memory, and a wireless network interface.

The processor can be configured to run and/or execute application modules, processes and/or functions associated with the device 100. For example, the processor can be configured to run and/or execute an image capture module that facilitates capturing and processing of an image produced by the electronic device 184. The processor can be, for example, a Field Programmable Gate Array (FPGA), an Application Specific Integrated Circuit (ASIC), a Digital Signal Processor (DSP), and/or the like. The processor can be configured to retrieve data from and/or write data to a memory device (not shown). As described herein, in some embodiments, the processor can cooperatively function with the network interface device and/or a radio to provide signals to communicatively couple the electronics module 132 to a remote computing device (e.g., such as the device 200 via wireless communication) and/or any other computing entity via a network. In some embodiments, the processor is a Bluetooth® low energy (BLE) processor, such as The Texas Instruments® CC2540 series of processors, the Broadcom® BCM43341 processor, and/or any other processor suitable or configured specifically to execute the Bluetooth® v4.0 low energy stack.

The memory (not shown) can be, for example, random access memory (RAM), memory buffers, hard drives, databases, erasable programmable read only memory (EPROMs), electrically erasable programmable read only memory (EEPROMs), read only memory (ROM), flash memory, hard disks, floppy disks, cloud storage, and/or so forth. In some embodiments, the memory stores instructions to cause the processor to execute modules, processes and/or functions associated the device 100. For example, the memory can store instructions to cause the processor to execute the image capture module.

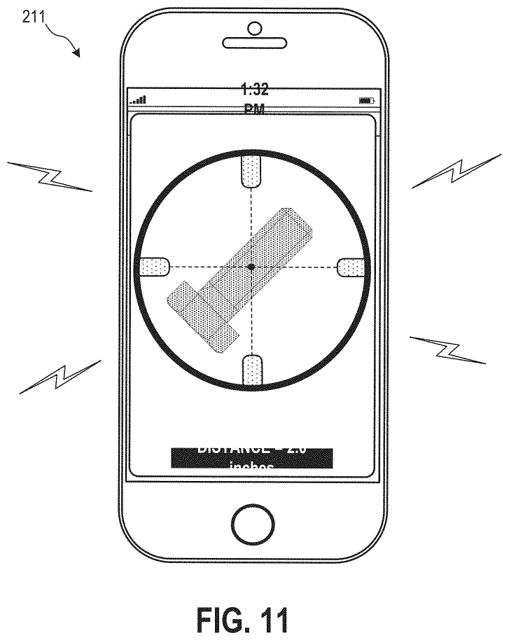

Referring to FIG. 8, the electronic controller 138 includes a wireless interface or radio 139, which can be any suitable communication device and can be a part of the overall processor architecture of the electronic control 138, (e.g., a part of a Bluetooth® processor). In other embodiments, the radio or wireless interface 139 can be distinct from a processor of the electronic control. In some embodiments, a short-range radio link can be established between the electronic module 132 and a mobile electronic device, such a mobile device 200 discussed below along with FIG. 11. For example, the electronic module 132 and/or the electronic controller 138 and the mobile device 200 can be paired via the Bluetooth® wireless protocol. Similarly stated, the electronic module 132 and/or the electronic control 138 and the mobile device 200 can be paired via a wireless protocol that facilitates the transmission of signals within a range of approximately 700 meters or less (i.e., a Class 3 radio) and/or having a frequency within the range of 2400 MHz and 2480 MHz. In such an embodiment, as described in further detail herein along with FIG. 11, the electronic module 132 and/or the electronic controller 138 can be operable to send and/or receive data from the mobile device 200 related to an image acquire by the device, such as from the electronic device 184.

Referring to FIG. 9, the proximal end portion 162 of the flexible member 160 is connected to the actuator 126 disposed within the proximal housing 112. Actuation of the proximal end portion of the flexible member moves the distal end portion of the flexible member 160 within the distal assembly 170, which permits deformation of each of the plurality of protrusion 192 extending from the distal end portion to flex outward into a gripping position.

In operation, actuation of the actuator 126 moves the mounting member 156 in the proximal housing 112 as discussed above to push the flexible member 160 forward within the flexible shaft 150 toward the distal housing 170. Movement of the flexible member 160 correspondingly moves the movable member 172 forward within the distal housing 171, which advances the protrusions 192 and the electronic device 184 forward within the distal housing 170. As the protrusions advance and extend outside of the outer housing 171, the protrusions flex or rotate outward to increase the size of a central region disposed between the tips 198 of the protrusion. In some embodiments, the advancement of the electronic device 184 also allows the central region produced between the tips 198 to be sensed by the electronic device 184 (e.g., viewed by the camera, in some embodiments). The endoscopic grabber device 110 can be advanced toward a target object (not shown) based on the camera view to place the target object within the central region. The user can release the actuator 126 to bias the flexible member 160 rearward and thereby collapse the tips 198 around the target object to grab the object. If the target object is magnetic, the optional magnet 190 can be used to grab the object via a magnetic connection alone or along with use of the protrusions 192.

Referring now to FIG. 11, a viewer device 211 is shown that can be used with an endoscopic grabber device, such as the example endoscopic grabber device 100 discussed above and/or example endoscopic grabber device 200 discussed below along with FIGS. 12A-12C. The viewer device 211 is shown as a portable phone device 211 or mobile device 211 (e.g., an iPhone®, an Android® device, a Windows® phone, a Blackberry® phone, etc.), but it is understood that various types of viewing devices can be used with endoscopic grabber devices discussed herein. Such viewing device can include, for example, a tablet computer (e.g., an Apple iPad®, a Samsung Nexus® device, a Microsoft Surface® device, etc.), or a computer (e.g., a laptop, desktop, smart TV, etc.), and/or any other suitable computing entity. In some embodiments, the viewer device 211 includes a mobile phone device 211 that has viewer application configured to connect with the electronic controller 138 of endoscopic grabber device 100 and display information received from the electronic device 184. The viewer application can be configured simply to display a view provided from the electronic controller 138 and/or the viewer application can be configured to modify the view as discussed further below, such as to add virtual line segments to provide a cross-hairs type view, show a virtual central point and/or to estimate distances to the tips 198 or the target object.

As shown in FIG. 11, in some embodiments, the viewer device 211 displays an image that is captured by the electronic device 184. The image can show the tips 198 of the protrusions 192 along with the target object and corresponding environmental features within its view while the endoscopic grabber device 100 is activated and being used. The electronic controller 138 can be configured to identify the tips 198 in the captured image. Alternatively, the viewer application can be configured to identify the tips 198 in the captured image, and the tips 198 could optionally be highlighted or marked to aid the identification. The viewer application or the electronic controller 138 can further be configured to identify the central point between the tips and a line segment between each tip and the central point, and to show the line segments and/or central point as virtual features on the display device 211. These virtual features could provide a cross-hairs type view to aid the user with aligning the endoscopic grabber device 100 effectively for grabbing the target object.

In addition, the electronic controller 138 and/or the viewer application can be configured to estimate distances to the target object and/or the distance that the protrusions 192 extend from distal end of the endoscopic grabber device 100. The size of the tips 198 can be known to the electronic controller 138 and/or the viewer application, which can be used to determine the distance that the tips are extended. Further, the electronic controller 138 and/or viewer application can compare the size of the target object being viewed with the size of the tips, as well as monitor the changing size of the object when approaching the target object, from which distances can be estimated. In addition, the distance that the protrusions are extended can be monitored based on movement of the movable member, for example, which can provide additional information for estimating distances and/or the size of the target object.

Referring now to FIGS. 12A to 12C along with FIG. 9, an embodiment of an endoscopic grabber 200 is shown in FIGS. 12A to 12C. Endoscopic grabber 200 generally includes the same aspects and features as endoscopic grabber 100 discussed above, except as discussed herein. Referring to FIG. 12B the endoscopic grabber 200 includes a flexible member 260 located within a flexible shaft 250 that extends between a manipulator portion 222 of the proximal housing 212 and the movable member 272 of the distal assembly 270. When the flexible shaft 250 is maintained in a general straight configuration between the proximal housing and the distal assembly, as shown in FIG. 12B, a length, Li, of the flexible member 260 is generally the same as that of the flexible shaft 250 between the endpoints of the flexible shaft (see also, the length Li shown in FIG. 9). As shown in FIG. 12A, however, the flexible shaft 250 is configured to have many different curvilinear arrangements as appropriate for following a path to gain access to a target object to grab, which changes the overall length of the flexible member 260 compared with the flexible shaft 250. Stated differently, bends and curves along the length of the flexible shaft 250 can induce tensile and compression forces, F, along the longitudinal axis of the flexible member 260, which can, under certain circumstances, cause the movable member 272 to bind within the distal assembly 270. Under such high bend circumstances, the actuation of an endoscopic grabber can become difficult due to the elongation of the flexible member 260.

Accordingly, the device 200 includes an actuator arrangement that allows for consistent actuation when the flexible shaft 250 is both straight and curved (in any amount). Referring to FIG. 12C, actuator 226 is configured to be adjustable with respect to flexible member 260 such that the flexible member 260 slidably engages an axial drive member 237 coupled to the longitudinal axis of the flexible member 226. When the actuator 226 rotates about the pivot 228, a drive lever 230 moves push ring 231 to translate along the longitudinal axis of axial drive member 237 and, thereby, move flexible member 260 toward the distal assembly 270. Axial drive member 237 can adjustably translate with respect to push ring or push plate 231 as compressive and tensile forces are encountered along the flexible member 260 responsive to curvilinear movements of the flexible shaft and flexible member. A user can further fine tune adjustment of the axial drive member 237 with respect to push ring 231 via angled release 235.

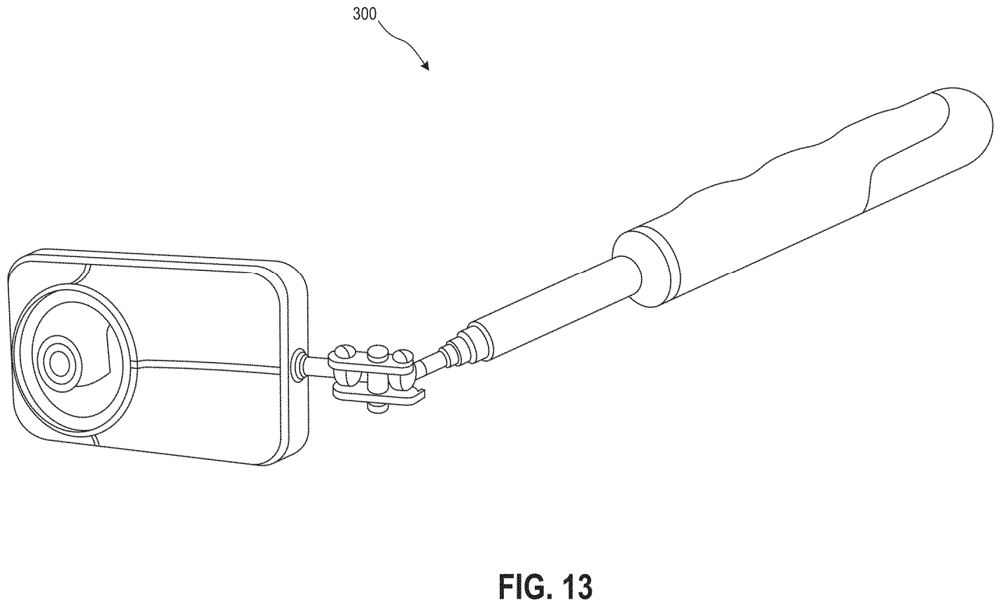

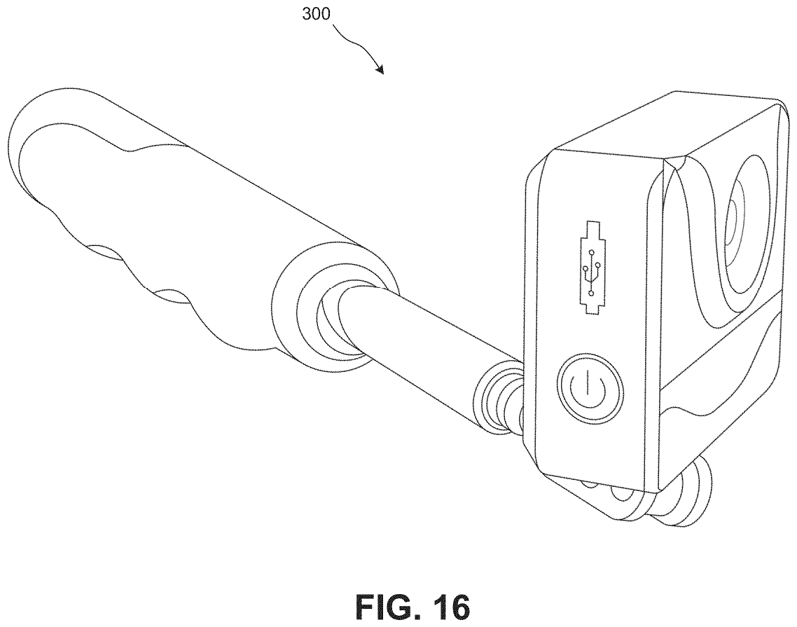

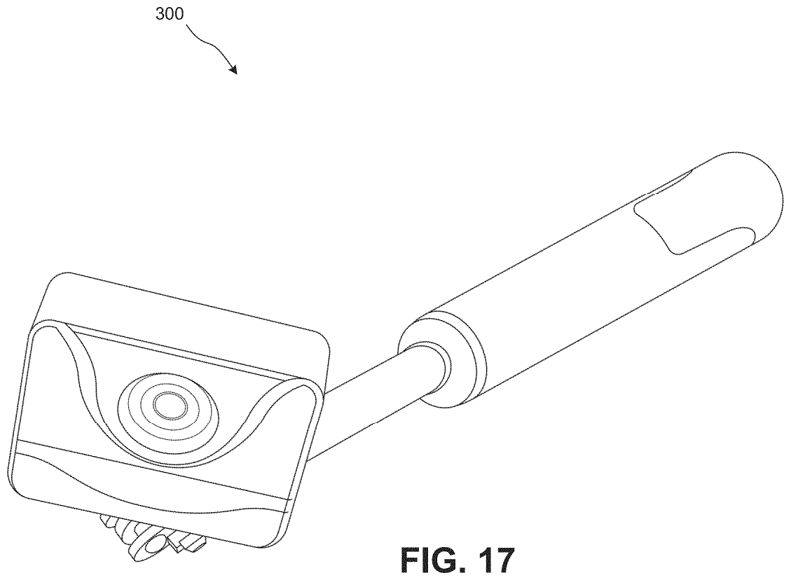

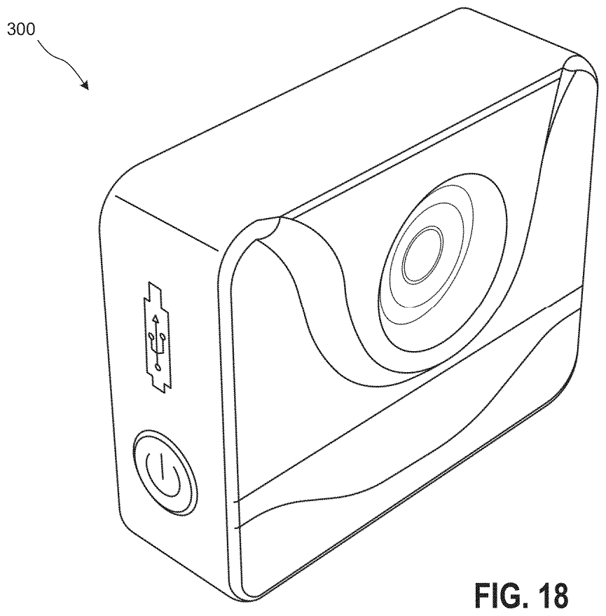

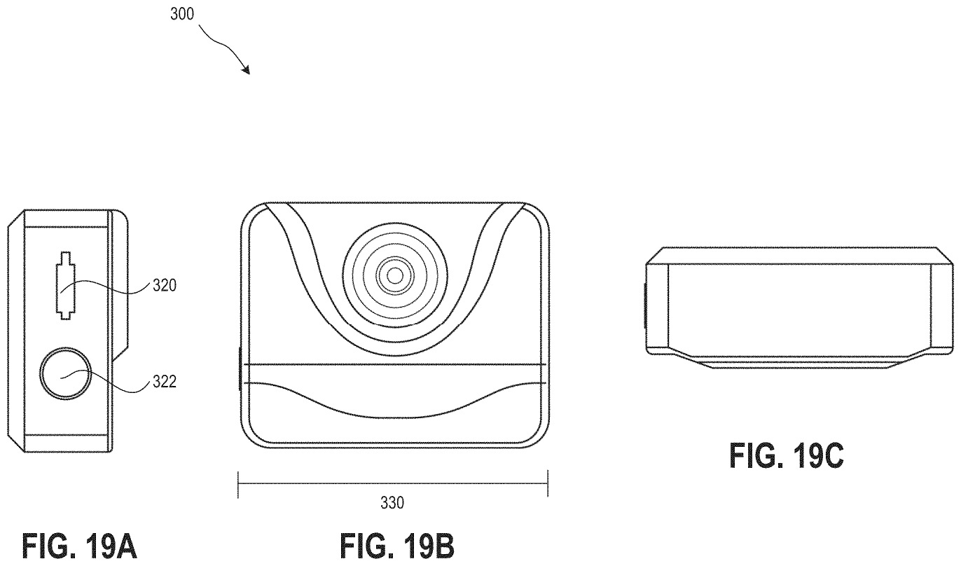

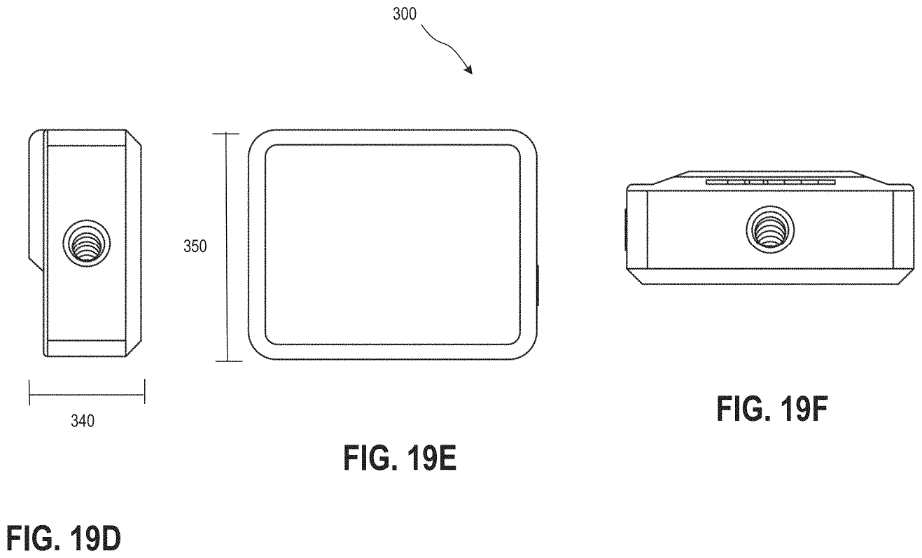

Referring now to FIGS. 13-19F, an extensible camera device 300 is shown. The extensible camera device 300 can be used on its own to view a target object or environment in a hard-to-reach or hard-to-view location, as well as in combination with an endoscopic grabber device, such as endoscopic grabber device 100. When used in combination with an endoscopic grabber device, the extensible camera device 300 can provide additional views and perspective for navigating to a target object to grab and/or for grabbing or manipulating the target object.

The extensible camera device 300 includes an extensible handle, an attached camera, and a flexible connection between a distal end of the extensible handle and the camera. The handle can be formed from concentric tubes of decreasing size disposed within each other to form an adjustable, extendable handle. Such an arrangement can allow the user to maintain and store the extensible camera device 300 in a compact configuration when not be used along with allowing the user to extend the length of the handle as needed for viewing a target object or environment. The extensible handle includes a grip portion at a proximate end, which allows the user to firmly hold the handle and manipulate the extensible camera device 300 during use.

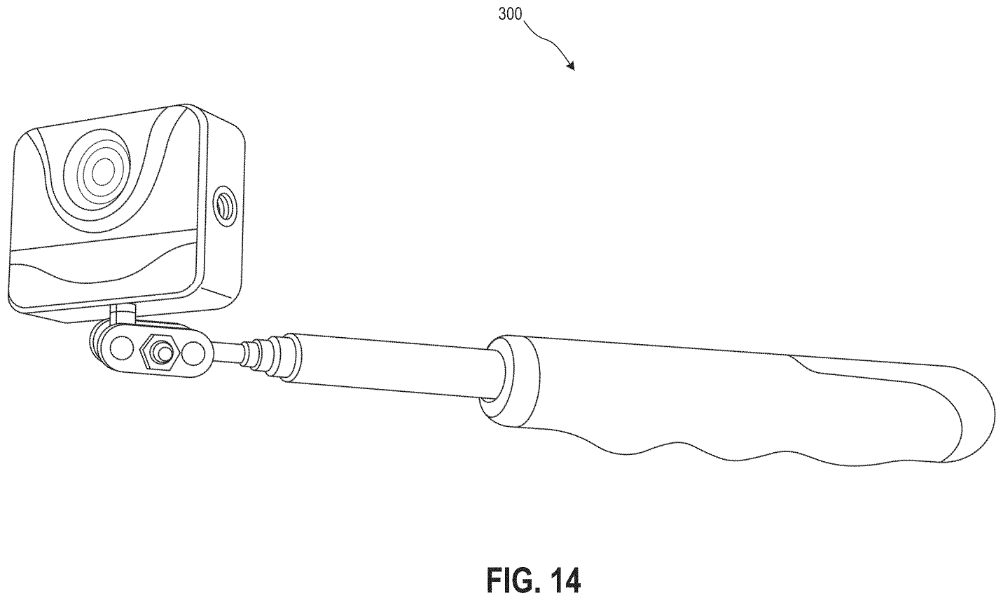

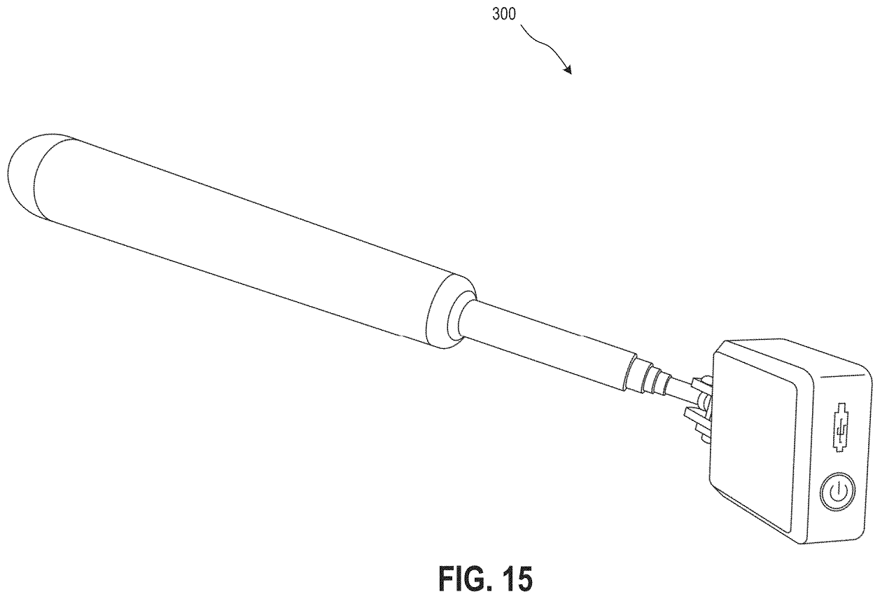

The camera is removably attached to distal end of the extensible handle, such as via a threaded attachment to securely retain the camera during use. The camera can include a camera that is specially designed for use with the extensible camera device 300, and can also include other readily available cameras. As such, the threaded connection at the distal end of the handle can be a standard size threaded connection for use with various cameras, such as a tripod type threaded connection. Further, the camera can include threaded attachments along multiple sides of the camera, such as at either a side portion as shown in FIGS. 13 and 15, or at a bottom portion as shown in FIGS. 14 and 16-17. Alternatively, the distal end of the extensible handle can include a mobile phone device attachment to connect a mobile phone device in a manner similar to the viewer mounting portion 116 discussed above along with the endoscopic grabber device 100 shown in FIGS. 1-10D and the viewer device 211 shown in FIG. 11. The camera can include internal storage for images captured thereby and a connection to retrieve the images, such as a USB connection. Further, the camera can include a slot (not shown) for a removable storage device to be installed and removed, such as a flash storage disk.

The flexible connection between the camera and the extensible handle is formed as a pair of adjacent hinges attached to the distal end of the extensible handle. One or both of the adjacent hinges can be formed as ball-in-socket hinges, which can allow a full range of motion between the camera and the extensible handle and for the orientation of the camera. As such, the extensible camera device 300 can permit the user to view objects and environments in wide range or orientations and directions during use.

While various embodiments of the invention have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

For example, although the electronic device 184 is shown as moving along with the movable member 172, in other embodiments, the electronic device 184 can remain stationary within the distal housing 170 when the movable member 172 moves. For example, in some embodiments, an endoscope can include an electronic device 184 that is fixedly mounted to an outer housing and does not move when the protrusions of a grabber extend from the device. In other embodiments, an endoscope can include an electronic device 184 that remains stationary relative to the protrusions during a first portion of the extension, and then moves along with the protrusion during a second portion of the extension.

Although the electronic device 184 is shown as being coupled to an electronic controller 139 via a wire, and the electronica controller is shown as transmitting a wireless signal to an image display device 211, in other embodiments, and suitable mechanisms for coupling the electronic device 184 to the display device 211 can be used. For example, in some embodiments, an electronic device mounted within a distal assembly can include a radio and can therefore be coupled directly to the display device without first being coupled to a controller. In other embodiments, a display device can be coupled to the controller via a wired coupling.

Although various embodiments have been described as having particular features and/or combinations of components, other embodiments are possible having a combination of any features and/or components from any of embodiments where appropriate. For example, although some embodiments are described as having a processor, a radio, a sensor, etc. disposed on a particular portion of a device, in other embodiments, any of the electronic circuit systems can be disposed on any suitable portion of an endoscopic device.

1. An apparatus comprising:
a proximal housing including an actuator;
a distal assembly including:
an outer housing;
an inner housing movably disposed within the outer housing, the inner housing defining a bore;
a plurality of protrusions coupled to an outer surface of the inner housing such that at least a portion of each of the plurality of protrusions is within a volume between the outer surface of the inner housing and an inner surface the outer housing; and
an electronic device coupled within the bore of the inner housing;
a flexible shaft having a distal end portion and a proximal end portion, the proximal end portion of the flexible shaft coupled to the proximal housing, the distal end portion of the flexible shaft coupled to the outer housing; and
a flexible member movably disposed within the flexible shaft, a distal end portion of the flexible member coupled to the inner housing, a proximal end portion of the flexible member coupled to the actuator, the actuator configured to move the flexible member to cause the inner housing to move within the outer housing between a first position and a second position, a distal end portion of each protrusion from the plurality of protrusions being in a first configuration within the outer housing when the inner housing is in the first position and in a second configuration outside of the outer housing when the inner housing is in the second position, a distal tip of each protrusion from the plurality of protrusions extending outside of the outer housing when the inner housing is in the second position.

2. The apparatus according to claim 1, wherein the distal tip of each protrusion from the plurality of protrusions extends outside of the outer housing by a first distance when the inner housing is in the first position, the distal tip of each protrusion from the plurality of protrusions extends outside of the outer housing by a second distance when the inner housing is in the second position, the second distance greater than the first distance.

3. The apparatus according to claim 1, wherein the electronic device is one of a light emitting device, a camera, or an ultrasonic device.

4. (canceled)

5. The apparatus of claim 1, wherein:
the plurality of protrusions is configured to operate together as a gripper, the gripper being in a closed position when the inner housing is in the first position and in an open position when the inner housing is in the second position; and
each protrusion of the plurality of protrusions is configured to deform when the inner housing moves between the first position and the second position.

6. The apparatus of claim 5, wherein:
the flexible shaft extending from the proximal end portion to the outer housing defines a longitudinal axis;
a proximal portion of each protrusion of the plurality of protrusions is coupled to the outer surface of the inner housing, each protrusion of the plurality of protrusions is oriented parallel with the longitudinal axis extending from the proximal portion to the corresponding distal end portion; and
the distal end portion of each protrusion of the plurality of protrusions is configured to radially deform away from the longitudinal axis when moving from the first configuration to the second configuration.

7. The apparatus of claim 6, wherein:
the distal tip of each protrusion of the plurality of protrusions is directed radially inward toward the longitudinal axis; and
the distal tip of each protrusion of the plurality of protrusions collectively define a central gripper region therebetween, the central gripper region located along the longitudinal axis.

8-9. (canceled)

10. The apparatus of claim 1, wherein:
the inner housing is configured to move along a stroke distance between the first position to the second position;
the plurality of protrusions being configured to move along with the inner housing through a first portion of the stroke distance, the inner housing configured to move relative to the plurality of protrusions through a second portion of the stroke distance; and
the electronic device is configured to move along with the inner housing through the stroke distance.

11. The apparatus of claim 10, wherein:
the inner housing is configured to move in a longitudinal direction along the stroke distance;
the plurality of protrusions is configured to move parallel to the longitudinal direction along with the inner housing for the first portion of the stroke distance;
the plurality of protrusions is configured to move parallel to the longitudinal direction along with the inner housing for the second portion of the stroke while the tip portion of each protrusion moves radially outward away from the longitudinal direction for the second portion of the stroke distance.

12. The apparatus of claim 10, wherein:
the inner housing is configured to move in a longitudinal direction along the stroke distance; and
the electronic device is configured to move parallel to the longitudinal direction along with the inner housing for the first portion of the stroke distance, the electronic device configured to remain at an intermediate position when the inner housing moves along the second portion of the stroke distance.

13-15. (canceled)

16. The apparatus according to claim 1, wherein the coupling of the proximal end portion of the flexible member to the actuator defines a slack-adjusting coupling, the slack-adjusting coupling configured to permit operation of the apparatus when the flexible shaft is in a straight orientation or in a curved orientation between the distal end portion and the proximal end portion of the flexible shaft, the slack-adjusting coupling configured to adjust a length between the actuator and the distal end portion of the flexible member such that the actuator is configured to be able to move the flexible member to cause the inner housing to move between the first and second positions when the flexible shaft is in the straight orientation and in the curved orientation.

17-18. (canceled)

19. An apparatus comprising:
a proximal housing;
a distal housing;
a flexible shaft having a distal end portion and a proximal end portion, the proximal end portion coupled to the proximal housing, the distal end portion coupled to the distal housing;
a flexible member within the flexible shaft having a distal end portion movably disposed within the distal housing and a proximal end portion coupled to an actuator disposed within the proximal housing, actuation of the proximal end portion of the flexible member is configured to move the distal end portion within the distal housing to deform a protrusion extending from the distal end portion; and
a camera coupled within the distal housing, the camera configured to receive an image including a portion of the protrusion.

20. The apparatus of claim 19, wherein the protrusion extends from the distal end portion in a longitudinal direction, and actuation of the proximal end portion is configured to radially deform a tip portion of the protrusion away from the longitudinal direction.

21. The apparatus of claim 20, wherein the actuator is configured to move in a linear direction within the proximal housing, actuation of the proximal end portion of the flexible member includes the actuator moving in the linear direction of at least a first distance, the linear movement of the at least a first distance is configured to radially deform the tip portion of the protrusion away from the longitudinal direction by a first angle of rotation.

22. The apparatus of claim 21, wherein the first angle of rotation is configured to be adjustable in comparison with the at least a first distance.

23. The apparatus of claim 21, wherein the actuation of the proximal end portion is configured to be a linear movement of up to a second distance that is greater than the at least a first distance, the linear movement of the up to a second distance is configured to radially deform the tip portion of the protrusion away from the longitudinal direction by up to a second angle of rotation that is larger than the first angle of rotation.

24. The apparatus of claim 23, wherein the up to a second angle of rotation is configured to be adjustable in comparison with the up to a second distance.

25. The apparatus of claim 19, wherein the apparatus includes a plurality of protrusions, each of the plurality of protrusions having a tip portion extending from the distal end portion in a corresponding longitudinal direction, the tip portions of the plurality of protrusions defining a central region therebetween, and actuation of the proximal end portion is configured to radially deform each tip portion of the plurality of protrusions in a radial direction away from central region.

26. The apparatus of claim 25, wherein each of the tip portions of the plurality of protrusions includes a tip at a distal end of the corresponding protrusion, each of the tips being directed inward toward the central region.

27-31. (canceled)

32. The apparatus of claim 25, wherein the plurality of protrusions is configured to cooperate to secure an object.

33. The apparatus of claim 32, wherein the tip portions of the plurality of protrusions are configured to secure the object.

34. (canceled)
